SEQUENCE LISTING (16 sequences)

SEQ ID NO: 1 - DNA, 54 bp, Gallus gallus
  CDS (1)..(54)
  sig_peptide (1)..(54)
atgaggtctttgctaatcttggtgctttgcttcctgcccctggctgctctgggg

SEQ ID NO: 2 - protein, 18 aa, Gallus gallus
MRSLLILVLCFLPLAALG

SEQ ID NO: 3 - protein, 29 aa, Bacillus brevis
MKKRRVVNSVLLLLLASALALTVAPMAFA

SEQ ID NO: 4 - protein, 18 aa, Drosophila melanogaster
MKAFIVLVALACAAPAFG

SEQ ID NO: 5 - protein, 23 aa, Saccharomyces cerevisiae
MLLQAFLFLLAGFAAKISAWSNM

SEQ ID NO: 6 - protein, 16 aa, Artificial
  L8LP  to test expression activity and secretion capacity for signal sequence L8LP when used with the present invention
MRLLLLLLLLLPAALG

SEQ ID NO: 7 - DNA, 63 bp, Apis mellifera
  CDS (1)..(63)
  sig_peptide (1)..(63)
atgaaattcttagtcaacgttgcccttgtttttatggtcgtgtacatttcttacatctatgcg

SEQ ID NO: 8 - protein, 21 aa, Apis mellifera
MKFLVNVALVFMVVYISYIYA

SEQ ID NO: 9 - DNA, 87 bp, Human immunodeficiency virus
  CDS (1)..(87)
  sig_peptide (1)..(87)
atgaaagtgaaggggaccaggaggaattatcagcacttgtggagatggggcaccttgctccttgggatgttgatgatctgtagtgct

SEQ ID NO: 10 - protein, 29 aa, Human immunodeficiency virus
MKVKGTRRNYQHLWRWGTLLLGMLMICSA

SEQ ID NO: 11 - DNA, 87 bp, Human immunodeficiency virus
  CDS (1)..(87)
  sig_peptide (1)..(87)
atgagagtgaaggggatcaggaagaattatcagcacttgtggagagggggcaccttgctccttgggatgttgatgatctgtagtgct

SEQ ID NO: 12 - protein, 29 aa, Human immunodeficiency virus
MRVKGIRKNYQHLWRGGTLLLGMLMICSA

SEQ ID NO: 13 - DNA, 1530 bp, Human immunodeficiency virus
atgaaagtgaaggggaccaggaggaattatcagcacttgtggagatggggcaccttgctccttgggatgttgatgatctgtagtgctacagaaaaattgtgggtcacagtttattatggagtacctgtgtggaaagaagcaactaccactctattttgtgcatcagatgctagagcatatgatacagaggtacataatgtttgggccacacatgcctgtgtacccacagaccccaacccacaagaagtagtattgggaaatgtgacagaaaattttaacatgtggaaaaataacatggtagaacagatgcaggaggatataatcagtttatgggatcaaagcctaaagccatgtgtaaaattaaccccactctgtgttactttaaattgcactgatttggggaaggctactaataccaatagtagtaattggaaagaagaaataaaaggagaaataaaaaactgctctttcaatatcaccacaagcataagagataagattcagaaagaaaatgcactttttcgtaaccttgatgtagtaccaatagataatgctagtactactaccaactataccaactataggttgatacattgtaacagatcagtcattacacaggcctgtccaaaggtatcatttgagccaattcccatacattattgtaccccggctggttttgcgattctaaagtgtaataataaaacgttcaatggaaaaggaccatgtacaaatgtcagcacagtacaatgtacacatggaattaggccaatagtgtcaactcaactgctgttaaatggcagtctagcagaagaagaggtagtaattagatctgacaatttcacgaacaatgctaaaaccataatagtacagctgaatgaatctgtagcaattaactgtacaagacccaacaacaatacaagaaaaagtatctatataggaccagggagagcatttcatacaacaggaagaataataggagatataagaaaagcacattgtaacattagtagagcacaatggaataacactttagaacagatagttaaaaaattaagagaacagtttgggaataataaaacaatagtctttaatcaatcctcaggaggggacccagaaattgtaatgcacagttttaattgtagaggggaatttttctactgtaatacaacacaactgtttaataatacatggaggttaaatcacactgaaggaactaaaggaaatgacacaatcatactcccatgtagaataaaacaaattataaacatgtggcaggaagtaggaaaagcaatgtatgcccctcccattggaggacaaattagttgttcatcaaatattacagggctgctattaacaagagatggtggtacaaatgtaactaatgacaccgaggtcttcagacctggaggaggagatatgagggacaattggagaagtgaattatataaatataaagtaataaaaattgaaccattaggaatagcacccaccaaggcaaagagaagagtggtgcagagagaaaaaagataa

SEQ ID NO: 14 - DNA, 1443 bp, Human immunodeficiency virus
  CDS (88)..(1443)
acagaaaaattgtgggtcacagtttattatggagtacctgtgtggaaagaagcaactaccactctattttgtgcatcagatgctagagcatatgatacagaggtacataatgtttgggccacacatgcctgtgtacccacagaccccaacccacaagaagtagtattgggaaatgtgacagaaaattttaacatgtggaaaaataacatggtagaacagatgcaggaggatataatcagtttatgggatcaaagcctaaagccatgtgtaaaattaaccccactctgtgttactttaaattgcactgatttggggaaggctactaataccaatagtagtaattggaaagaagaaataaaaggagaaataaaaaactgctctttcaatatcaccacaagcataagagataagattcagaaagaaaatgcactttttcgtaaccttgatgtagtaccaatagataatgctagtactactaccaactataccaactataggttgatacattgtaacagatcagtcattacacaggcctgtccaaaggtatcatttgagccaattcccatacattattgtaccccggctggttttgcgattctaaagtgtaataataaaacgttcaatggaaaaggaccatgtacaaatgtcagcacagtacaatgtacacatggaattaggccaatagtgtcaactcaactgctgttaaatggcagtctagcagaagaagaggtagtaattagatctgacaatttcacgaacaatgctaaaaccataatagtacagctgaatgaatctgtagcaattaactgtacaagacccaacaacaatacaagaaaaagtatctatataggaccagggagagcatttcatacaacaggaagaataataggagatataagaaaagcacattgtaacattagtagagcacaatggaataacactttagaacagatagttaaaaaattaagagaacagtttgggaataataaaacaatagtctttaatcaatcctcaggaggggacccagaaattgtaatgcacagttttaattgtagaggggaatttttctactgtaatacaacacaactgtttaataatacatggaggttaaatcacactgaaggaactaaaggaaatgacacaatcatactcccatgtagaataaaacaaattataaacatgtggcaggaagtaggaaaagcaatgtatgcccctcccattggaggacaaattagttgttcatcaaatattacagggctgctattaacaagagatggtggtacaaatgtaactaatgacaccgaggtcttcagacctggaggaggagatatgagggacaattggagaagtgaattatataaatataaagtaataaaaattgaaccattaggaatagcacccaccaaggcaaagagaagagtggtgcagagagaaaaaagataa

SEQ ID NO: 15 - protein, 451 aa, Human immunodeficiency virus
AYDTEVHNVWATHACVPTDPNPQEVVLGNVTENFNMWKNNMVEQMQEDIISLWDQSLKPCVKLTPLCVTLNCTDLGKATNTNSSNWKEEIKGEIKNCSFNITTSIRDKIQKENALFRNLDVVPIDNASTTTNYTNYRLIHCNRSVITQACPKVSFEPIPIHYCTPAGFAILKCNNKTFNGKGPCTNVSTVQCTHGIRPIVSTQLLLNGSLAEEEVVIRSDNFTNNAKTIIVQLNESVAINCTRPNNNTRKSIYIGPGRAFHTTGRIIGDIRKAHCNISRAQWNNTLEQIVKKLREQFGNNKTIVFNQSSGGDPEIVMHSFNCRGEFFYCNTTQLFNNTWRLNHTEGTKGNDTIILPCRIKQIINMWQEVGKAMYAPPIGGQISCSSNITGLLLTRDGGTNVTNDTEVFRPGGGDMRDNWRSELYKYKVIKIEPLGIAPTKAKRRVVQREKR

SEQ ID NO: 16 - DNA, 738 bp, Baculovirus
atgccggattattcataccgtcccaccatcgggcgtacctacgtgtacgacaacaagtactacaaaaatttaggtgccgttatcaagaacgctaagcgcaagaagcacttcgccgaacatgagatcgaagaggctaccctcgaccccctagacaactacctagtggctgaggatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgcgttag

(19) United States
(12) Patent Application Publication
(10) Pub. No.: US 2003/0170619 A1
(43) Pub. Date: Sep. 11, 2003

(54) Nucleic acid capable of promoting gene expression

(76) Inventor: Toshio Hara, Fukuoka (JP)

Correspondence Address: COVINGTON & BURLING, ATTN: PATENT DOCKETING, 1201 PENNSYLVANIA AVENUE, N.W., WASHINGTON, DC, 20004-2401, US

(21) Appl. No.: 10/361,849

(22) Filed: Feb. 11, 2003

Related U.S. Application Data
(60) Provisional application No. 60/355,752, filed on Feb. 12, 2002.

(51) Int. Cl. C12Q 1/70; C07H 21/02; C12N 15/867; C12N 5/06; C12N 15/86

(52) U.S. Cl. 435/5; 435/456; 435/348; 435/320.1; 536/23.72

(57) ABSTRACT

Provided is a nucleic acid capable of inducing or promoting the expression of a gene. The present invention relates to a nucleic acid capable of inducing or promoting the expression of a gene. The present invention provides, among other things, a secretory signal sequence of a chicken lysozyme or a secretory signal sequence of an SF162 type HIVgp120. When the SF2 type HIVgp120 has an intrinsic signal sequence, it is difficult to artificially express the gene. The expression thereof, however, can be induced by replacing the intrinsic signal sequence with the two secretory signal sequences.

This specification includes a Sequence Listing, comprising 14 pages, found after the Abstract.

### BACKGROUND OF THE INVENTION

1. Field of the Invention

The present invention relates to a nucleic acid instrumental in the enhanced expression of a gene, particularly to a nucleic acid capable of promoting the production of a transcriptional product from the gene and promoting the production of a translational product therefrom. The present invention further relates to an expression vector, or similar moiety, of an SF2 type HIVgp120 expressed by using such a nucleic acid. The present invention also relates to a method and system for detecting such nucleic acid capable of promoting the expression of a gene.

2. Description of the Related Art

Not only does a gene preserve the genetic character of living organisms in future generations in the form of genome reproduction, it also expresses functional gene products such as RNA and protein in accordance with a program contained in the information which the gene itself possesses. The expression of the gene product from such gene is gene expression, and, in principle, includes transcription for creating RNA from a gene and translation for creating protein from RNA. Pursuant to such gene expression, the formation or organization of various molecular complexes, cell organelles, cells, tissues or the like in living organisms is realized, which further enables living organisms to adapt to a diversified environment.

This gene expression is highly controlled relative to time, environment, and other factors. For instance, as control elements in a transcription, there are cis elements (for example, a promoter or operator on the gene) and trans elements (for example, an activator having affinity to such cis factor), among others. Similarly, there are elements that promote and control translation. Such control elements express genes in vitro and in vivo and are effective in artificially producing expression products. Thus, research has been conducted on the control elements of these gene expressions, and numerous control elements have been discovered heretofore.

Meanwhile, the protein ultimately expressed under the aforementioned control must be transferred to the necessary places within the living body for the formation of tissue and other functions. As an element instrumental in the transfer of protein, the existence of a signal peptide has been conventionally known. This signal peptide is formed on the N terminal of the pre-protein. And, pursuant to this signal peptide, the ultimate protein is transported to an appropriate place by being secreted outside the cell, bound with a membrane, or transferred inside the nucleus.

For example, it has been indicated that by introducing a honeybee melittin signal peptide into a protein, the secretion of such protein produced inside the cell can be promoted (GENE 98: 177-183 (1991)), and a protein synthesizing system employing the above has been put into practical application (“pMelBac” high-level secretion type expression vector, Invitrogen).

Before the present invention, only the signal peptide of bee poison had been reported as a signal peptide capable of promoting the secretion of protein, and it was unclear as to whether other signal peptides possessed similar activity.

Moreover, it was thought that a conventional bee poison signal peptide would promote the secretion of protein. In other words, conventionally, it was thought that the transcriptional adjustment element would adjust the transcription process, the translational adjustment element would adjust the translation process, and the signal peptide would induce the transfer of the ultimate product. Thus, the gene coding the signal peptide (hereinafter referred to as the signal sequence) was not considered to possess any activity other than post-translational modification and transfer of protein.

### SUMMARY OF THE INVENTION

During the course of conducting research employing a chicken lysozyme secretory signal and an SF162 type HIVgp120 secretory signal, the present inventors discovered that these secretory signals are also capable of promoting the secretion of protein, and further found that such secretory signal sequences are also capable of adjusting the transcription, and thereby achieved the present invention.

In other words, one embodiment of the present invention is to employ the aforementioned signal sequence for implementing or promoting the expression of a gene, including but not limited to structural genes.

Specifically, an embodiment of the present invention is a nucleic acid inserted into the upstream of an arbitrary gene linked expressibly to a promoter and capable of implementing or promoting the expression of the gene from the promoter, wherein the nucleic acid is a secretory signal sequence of a chicken lysozyme or a secretory signal sequence of an SF162 type human immunodeficiency virus (HIV)gp120.

According to said embodiment of the present invention, even if the gene expression can hardly be observed or the expression level is low, by linking the aforementioned signal sequence to the upstream of the target gene, the implementation of such gene expression or the promotion of the expression level thereof may be realized.

Here, “gene expression” shall include transcription, translation, post-translational modification, and post-modification protein secretion.

Moreover, the secretory signal sequence of the chicken lysozyme is, for example, the sequence shown in SEQ ID NO. 1, and the secretory signal sequence of the SF162 type human immunodeficiency virus (HIV) gp120 is, for example, the sequence shown in SEQ ID NO. 11.

An alternative embodiment of the present invention is an expression cassette for implementing or promoting the expression of a gene, comprising a promoter connected expressibly to the gene; a secretory signal sequence disposed upstream of the gene and expressed integrally with the gene from the promoter; and a 3′UTR disposed downstream of the gene; wherein the secretory signal sequence is a secretory signal sequence of a chicken lysozyme or a secretory signal sequence of an SF162 type human immunodeficiency virus (HIV) gp120.

By providing this type of expression cassette and connecting it to a desired gene, a gene product (for example, a transcriptional product such as mRNA or a translational product such as protein) may be obtained with ease.

In an embodiment of the present invention, the expression cassette may comprise a promoter and 3′UTR deriving from baculovirus polyhedrin It is thereby possible to increase the transcription activity by employing a promoter deriving from baculovirus polyhedrin.

In alternative embodiments of the present invention, one may include the expression cassette in a vector and provide it as an expression vector. By including the expression cassette in a vector, an auto-reproduction function can be provided thereto. Thus, the gene expression may be conducted stably by inserting and preserving the expression vector, to which the gene is inserted, in a suitable host cell. A baculovirus vector, for example, may be employed as such a vector When using an expression vector employing such baculovirus vector, the production of a desired gene product, mRNA, protein and so on may be conducted more easily by providing such vector together with the insect cell as the host cell.

Moreover, another embodiment of the present invention is the implementation or promotion of the expression of an SF2 type HIVgp120, the expression of which was conventionally difficult, by employing the aforementioned secretory signal sequences.

Specifically, in an embodiment of the present invention, an SF2 type HIVgp120 expression cassette, comprises: an SF2 type HIVgp120; a secretory signal sequence linked to the upstream of the SF2 type HIVgp120; a promoter connected to the upstream of the SF2 type HIVgp120 with the secretory signal sequence therebetween so as to express the SF2 type HIVgp120 to which the secretory signal sequence is linked; and a 3′UTR connected to the downstream of the SF2 type HIVgp120, wherein the secretory signal sequence is a secretory signal sequence of a chicken lysozyme or a secretory signal sequence of an SF162 type human immunodeficiency virus (HIV) gp120.

With respect to the SF2 type HIVgp120, although the expression of gp120 cannot be observed even upon connecting this gene to an expression vector comprising a unique signal sequence, by replacing it with the aforementioned secretory signal sequences, the implementation or promotion of the expression thereof becomes possible.

The aforementioned SF2 type HIVgp120 expression cassette also may be incorporated in a vector and provided as an SF2 type HIVgp120 expression vector. Moreover, the SF2 type HIVgp120 expression cassette or the SF2 type HIVgp120 expression vector incorporated in a vector may be introduced into a cell, preferably an insect cell, and provided as an SF2 type HIVgp120 expression cell.

According to the foregoing structures, the production of an SF2 type HIVgp120 protein has been facilitated, and the SF2 type HIVgp120 protein expressed from such expression cell or the like possesses a similar bioactivity as a natural SF2 type HIVgp120 protein and binding ability with a CD4+T cell, and it is therefore expected to contribute to the development of vaccines and antibodies.

Yet another embodiment of the present invention utilizes the discovery that a well-known secretory signal sequence possesses activity capable of promoting the expression of a gene, and particularly that the promotion activity of such gene expression results at least from the promotion at the transcription level.

In other words, one embodiment of the present invention is a method for determining whether a secretory signal sequence, which is a test sample, is capable of implementing or elevating the expression of a gene, which method comprises: an expression vector preparation step wherein an expression vector is prepared by connecting a monitor gene, to which the secretory signal sequence is linked upstream so as to enable the expression from the promoter; a cell introduction step for introducing the expression vector into the cell; and a measurement step for measuring the representation of the monitor gene in the cell, wherein the representation is measured by measuring the transcriptional product of the monitor gene in the measurement step.

An alternative embodiment of the present invention is a system for determining whether a secretory signal sequence, or other test sample nucleic acid, is capable of elevating the expression of a gene, the determination system comprising (i) an expression vector having a promoter and a 3′UTR for attaching a monitor gene coupled to an upstream secretory signal sequence (or other test sample nucleic acid) in a manner permitting expression of the secretory signal sequence (or other test sample nucleic acid) by using the promoter and (ii) a cell for expressing the monitor gene, into which cell has been introduced the expression vector to which the monitor gene is attached, wherein the determination system determines whether the secretory signal sequence (or other test sample nucleic acid) is capable of elevating the expression of the monitor gene, as measured by representation of the monitor gene in the cell.

Generally speaking, an antibody must be used in order to detect a specific protein from the likes of cell secretions. Further, production time and cost are required for the preparation of this antibody. Meanwhile, the detection of a transcriptional product may be conducted by employing the likes of synthesized DNA, which is relatively easy to obtain or create. Thus, according to the foregoing structure, the gene expression promotion activity in the secretory signal sequence, which is a test sample, may be determined with ease.

### BRIEF DESCRIPTION OF THE DRAWINGS

FIG. 1 is a diagram showing the construction process of the vector in which the secretory signal sequence is inserted into the upstream of a lysozyme gene in Example 1;

FIG. 2 is a graph showing the results of the production amount, measured over time, of intracellular (A) and extracellular (B) lysozyme protein pursuant to the respective secretory signal sequences in Example 1;

FIG. 3 is a diagram showing the construction process of the expression vector for measuring the gene expression promotion activity in the secretory signal sequence employing the SF2 type HIVgp120 gene in Example 2;

FIG. 4 is a diagram showing the electrophoretic photographs upon determining the existence/non-existence of expression secretory activity of the SF2 type HIVgp120 in the respective secretory signal sequences in Example 3;

FIG. 5 is a diagram showing the electrophoretic photographs upon determining whether the respective SF2 type HIVgp120 proteins expressed and secreted pursuant to the addition of a secretory signal sequence is sugar-modified upon processing the proteins with glycosidase in Example 4;

FIG. 6 is a diagram showing the results of flow cytometry measurements showing that the binding ability to the CD4+T cell of the respective SF2 type HIVgp120 proteins expressed and secreted pursuant to the addition of a secretory signal sequence is similar to a natural SF2 type HIVgp120 protein in Example 5; and

FIG. 7 is an electrophoretic diagram showing that the respective SF2 type HIVgp120mRNA expressions have been induced pursuant to the addition of a secretory signal sequence in Example 6.

### DETAILED DESCRIPTION

Illustrative embodiments of the present invention are now described.

First Set of Illustrative Embodiments: Nucleic Acids Capable of Promoting Gene Expression

An embodiment of the present invention is a nucleic acid capable of promoting the gene expression, wherein the nucleic acid is a secretory signal sequence instrumental in the post-translational modification and transportation of protein, such as a chicken lysozyme secretory signal sequence or an SF162 type HIVgp120 secretory signal sequence.

These secretory signal sequences were believed to be instrumental in post-translational processes, such as post-translational modification or processing, but the present inventors discovered that such secretory signal sequences are also capable of adjusting, implementing or promoting the process of gene transcription.

Therefore, in addition to implementing or promoting the process of translation for producing protein, an embodiment of the present invention is a nucleic acid that may also be used for implementing or promoting the transcription process for producing RNA.

The nucleic acid of the present invention may be a secretory signal sequence, and may be used for the purpose of secreting the protein produced by translation outside the cell. As described above, protein may easily be purified through extracellular secretion of the protein.

The aforementioned chicken lysozyme secretory signal sequence is shown in SEQ ID NO. 1, and the secretory signal peptide thereof is shown in SEQ ID NO. 2. Further, the SF162 type HIVgp120 secretory signal sequence is shown in SEQ ID NO. 11, and the secretory signal peptide thereof is shown in SEQ ID NO. 12.

These sequences may be altered within a scope enabling the maintenance of the expression activity of the aforementioned gene. Replacement, deletion, addition and so on may be included in such alteration. The altered nucleic acid sequence is included in the “nucleic acid capable of implementing or promoting gene expression” according to the present invention so long as it possesses activity for implementing or promoting the expression of a gene.

The production of the aforementioned chicken lysozyme secretory signal sequence may be directly obtained from a chicken chromosome or, more simply, synthesized with a DNA synthesizer based on the aforementioned SEQ ID NO. 1.

Similarly, the SF162 type HIVgp120 secretory signal sequence may also be obtained from the chromosome gene of an SF162 type HIV, or, more simply, synthesized pursuant to synthesized DNA based on the aforementioned SEQ ID NO. 11.

In other embodiments of the present invention, in order to implement and promote the expression of a gene with such secretory signal sequences, the secretory signal sequence is linked to the upstream of the target structural gene. Upon this link, the reading frame of the secretory signal sequence becomes identical with the reading frame of the structural gene during the transcription from the promoter, and implemented such that it is transcribed integrally with the structural gene.

An expression cassette comprising such secretory signal also may be structured for implementing or promoting the expression of the gene with the secretory signal sequence as described above.

In the structure of alternative embodiments of the expression cassette of the present invention, the secretory signal sequence is linked to the upstream of a desired structural gene, and this is disposed at the downstream of the promoter. In other embodiments, a 3′UTR may be disposed at the downstream of the structural gene.

The promoter used in the expression cassettes is not particularly limited, and may be arbitrarily selected and used so as long as it functions as a promoter in the host cell to which such expression cassette is introduced. Moreover, it is preferable to use a promoter having a high expression level. In addition, the UTR, for example, may derive from the same origin as the promoter. For instance, as the promoter or UTR which functions in an insect cell, a baculovirus polyhedrin promoter (SEQ ID NO. 16l ) may be used.

Further, other nucleic acid sequences may be provided to the flanking region of the aforementioned expression cassette so long as they do not influence the implementation or promotion of the expression of the aforementioned structural gene. For example, an expression vector may be structured preferably by providing a vector sequence to the flanking region.

Since it is possible to enable auto-reproduction by providing a vector sequence, this is effective in the amplification of the expression cassette or introduction into the host cell. In addition, by employing a vector capable of being reproduced in the host cell, the expression of the structural gene connected to the expression cassette may be implemented inside the host cell stably and continuously.

The vector used herein is not particularly limited, and a vector possessing an auto-reproduction function in a prokaryote may also be used. By using a vector possessing an auto-reproduction function in a prokaryote as described above, an expression cassette may be reproduced inside the prokaryote easily and quickly. As this type of vector, for example, a phage vector may also be used in addition to a plasmid vector such as pRB322 or pUC.

Moreover, as a vector in a eukaryote, for example, a virus vector such as a baculovirus or SV40 may be used. Preferably selected is a vector having a reproductive function in the host cell to which such expression vector is introduced. For instance, when an insect cell is the host cell, a baculovirus vector may be used. In addition, this expression vector, together with the host cell, may be provided as an expression kit. By connecting the structural gene to the downstream of the secretory signal sequence provided to this expression vector, gene expression can be implemented even more simply, and an expression product may be obtained easily as a result thereof.

Second Set of Illustrative Embodiments: Expression of SF2 type HIVgp120

1. SF2 type HIV gp120 Expression Nucleic Acid

The SF2 type HIVgp120 is an HIV possessing directivity toward a CD4+T cell, and, meanwhile, the aforementioned SF162 type HIV is an HIV possessing directivity toward a macrophage. (J. Virol., Vol. 64, p4390-4398 (1990), Nature, Vol. 349, p167-169 (1991)).

With this type of directivity toward a target cell, it has been discovered that the glycoprotein 120 (gp120) of HIV is instrumental thereto. Therefore, this HIVgp120 protein is expected to be an effective material in the defense against HIV.

Nevertheless, since it is difficult to artificially express an SF2 type HIVgp120 protein, it was not possible to artificially produce the gp120 protein.

In an embodiment of the present invention, the aforementioned chicken lysozyme secretory signal sequence and SF162 type HIVgp120 secretory signal sequence may be used in implementing or promoting the SF2 type HIVgp120 expression.

In another embodiment, in order to implement or promote the SF2 type HIVgp120 expression with the aforementioned secretory signal sequences, it is possible to replace the unique secretory signal sequence provided to the upstream of the SF2 type HIVgp120 with an SF162 type secretory signal sequence deriving from the aforementioned chicken. Further, SEQ ID NO. 13 shows a cDNA sequence of an SF2 type HIVgp120 comprising a unique secretory signal sequence. Moreover, SEQ ID NO. 14 shows a cDNA sequence of an SF2 type HIVgp120 in which the unique signal sequence was deleted, and SEQ ID NO. 15 shows the amino acid sequence thereof.

Accordingly, the chicken lysozyme origin or the SF162 type HIVgp120 secretory signal sequence shown in SEQ ID NO. 1 or SEQ ID NO. 11 may be provided to the upstream of the SF2 type HIVgp120 in which the signal sequence was deleted.

In order to actually express the SF2 type HIVgp120 with the secretory signal sequence replaced, an embodiment of the present invention having the foregoing structure may be inserted into the expression vector.

This vector is not particularly limited, and may be selected in accordance with the purpose thereof. For example, since the gp120 is a glycoprotein, a vector that is capable of being reproduced in the host cell that can be sugar-modified may be used. As this type of host cell, for example, an insect cell SF21 or the like may be used, and, when selecting the insect cell as the host, a baculovirus vector may be used as the vector.

A promoter, UTR or the like is provided on the expression vector, and such promoter, UTR or the like may be selected, similar to the vector, so long as it is able to function as a host cell. For instance, a baculovirus polyhedrin promoter having high expression activity may be preferably used upon employing the aforementioned insect cell as the host. Further, in correspondence therewith, a UTR deriving from polyhedrin may be used.

The baculovirus vector comprising such polyhedrin promoter or UTR is commercially available as “pVL1392” and “pVL1393” (Invitrogen), and may be used. In this pVL1392 or 1393, a multicloning site is provided to the downstream of the polyhedrin promoter. Thus, an expression vector can be produced by inserting the SF2 type HIVgp120, which comprises a signal sequence deriving from the aforementioned chicken lysozyme or an SF162 type HIV secretory signal sequence, into this cloning site.

2. SF2 type HIVgp120 Expression Cell

In another embodiment of the present invention, to stably and continuously express the SF2 type HIVgp120 and obtain a gp120 expression product, it is preferable to introduce the aforementioned expression vector into the host cell and create an expression cell.

For example, with the expression vector employing the foregoing baculovirus vector, such expression vector is introduced into the baculovirus with the lipofectin method or the like in order to create a recombinant virus. Then, by infecting the insect cell with this recombinant virus, the aforementioned expression vector may be introduced into the insect cell.

And, an SF2 type HIVgp120 mRNA is produced inside the created expression cell. Moreover, outside the cell, it is possible to secrete the SF2 type gp120 glycoprotein retaining the binding ability to the CD4+T cell, which a natural SF2 type gp120 possesses.

Therefore, by employing this expression cell, mass production of the SF2 type gp120 is possible, and this may be used as a material for defense against HIV, such as vaccine for HIVgp120.

Third Set of Illustrative Embodiments: Methods of Determinating Gene Expression Promotion Activity in Secretory Signal Sequence

The present inventors have discovered that the transcription of a gene is implemented or promoted pursuant to a secretory signal sequence, and that the expression of the gene is implemented or promoted thereby. Thus, by monitoring the transcription level of the gene comprising the secretory signal sequence, it is possible to determine whether such secretory signal sequence is capable of implementing or promoting the expression of a gene, including translation and transcription.

1. Preparation of Expression Vector

In an embodiment of the present invention, to perform the determination, the secretory signal sequence, which is a test sample, is linked to the upstream of the monitor gene, and this group is inserted between the promoter and 3′UTR on the expression vector in order to construct the expression vector.

In some embodiments, the monitor gene may be a gene which will not, or will only with difficulty, implement the expression of a gene, for example, the cDNA (SEQ ID NO. 14) of the SF2 type HIVgp120 or the like shown in the second embodiment.

Although a baculovirus comprising a polyhedrin promoter or 3′UTR may be used in some embodiments, the vector is not limited thereto.

In other embodiments of the present invention, for example, as a positive control of this determination method, the secretory signal sequence of the SF162 type HIVgp120 or the secretory signal sequence of the chicken lysozyme may be used. Meanwhile, as a negative control thereof, the secretory signal sequence of the SF2 HIVgp120 may be used.

As described above, by respectively linking the secretory signal of the test sample and the secretory signal sequence of the control to the upstream of the monitor gene, and connecting them expressibly to the downstream of the aforementioned expression vector promoter, the test sample and the expression vector of the control are created thereby.

2. Introduction of Expression Vector into Cell

In an embodiment of the present invention, after the expression vector is created, such expression vector is inserted into the cell.

Various suitable methods may be employed, depending on the type of vector or cell, for the introduction of this expression vector inside the cell. For example, in the case of the aforementioned baculovirus vector, the expression vector may be introduced into the baculovirus with the lipofectin method or the like. And, this recombinant baculovirus may be inserted into an insect cell, for example, an SF21 cell, by infecting the insect cell with the baculovirus.

3. Measurement of Representation

In one embodiment of the present invention, representation of the foregoing monitor gene is measured in the cell to which the expression vector is introduced. The measurement of this representation may also be based on, among other things, the production amount of the protein, which is a translational product, or may be based on the production amount of the mRNA, which is a transcriptional product.

Further, these may be combined. That is, in some embodiments, the production amount of the transcriptional product may be measured first, and the production amount of the translational product may be measured thereafter. For instance, the aforementioned activity of the secretory signal sequence may be measured based on the production amount of the transcriptional product, and the extracellular secretion may be further determined based on the translational product.

In an embodiment of the present invention, in order to measure the production amount of the translational product, nucleic acid is extracted from the cells to which the respective expression vectors were introduced. Then, the mRNA against the monitor gene in the extracted nucleic acid can be detected pursuant to the Northern blotting method. In addition, the extraction of the nucleic acid and the Northern blotting method may be conducted by employing the method of Maniatis, et al. (Molecular & Cloning, Cold Spring Harbour Press) or other methods.

Moreover, the translational product may be detected, for example, by employing a specific antibody in the translational product (protein) through an immunological method such as Western blotting, ELISA, and other methods.

In another embodiment, by enabling the measurement of the gene expression promotion activity in a secretory signal sequence pursuant to the production amount of the transcriptional product, it becomes possible to determine the foregoing activity even in cases where the protein, which is a translational product, does not possess a specific antibody. Furthermore, in general cases where a specific antibody is present, it is possible to measure the gene expression promotion activity in the aforementioned secretory signal sequence without having to use costly materials as described above.

### EXAMPLES

The present invention is now explained in detail with reference to the Examples, but the present invention is in no way limited thereto.

Example 1: Expression and Elevation of Chicken Lysozyme

The plasmid pVL1393Lyz I is structured based on pVL1393 (Invitrogen) , and a lysozyme gene deriving from chicken is inserted therein, and a signal sequence (SEQ ID NO. 5) deriving from yeast invertase is inserted in the upstream thereof. Although the lysozyme gene possessing this signal sequence enables the expression in coliform bacillus and yeast, the expressed protein forms the inclusion without being secreted. Thus, the aforementioned plasmid was used to attempt the expression and secretion with an insect cell, but the protein did not secrete, and the expression was also extremely low.

Therefore, the signal sequence on the plasmid was replaced with various signal sequences, and the existence of the expression activity and secretion capacity was examined.

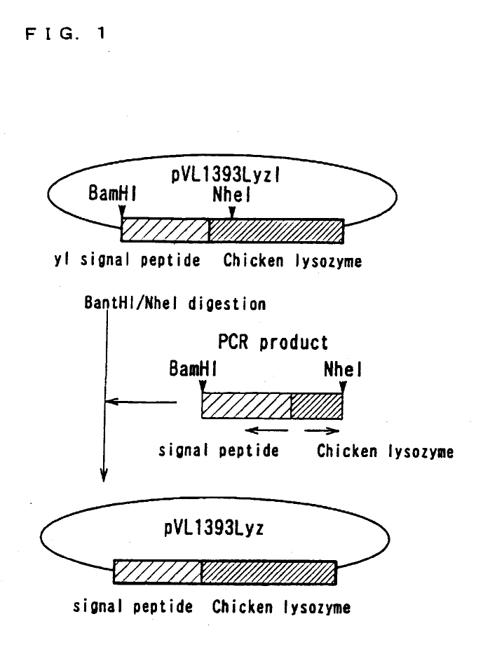

Moreover, the signal sequence used here is shown in Table 1, and the signal sequences and signal peptides thereof are shown in the sequence table (SEQ ID NOS. 1 and 2: chicken lysozyme signal sequence/peptide; SEQ ID NO. 3: bacillus brevis CWP signal sequence/peptide; SEQ ID NO. 4: fruit fly lysozyme signal sequence/peptide; SEQ ID NO. 6: L8LP signal sequence/peptide; SEQ ID NOS. 7 and 8: honeybee melittin signal sequence/peptide) . The replacement procedure of these signal sequences is shown in FIG. 1.

TABLE 1

AMINO ACID SEQUENCE OF RESPECTIVE SIGNALS

| | |
|---|---|
| ORIGIN | CHICKEN LYSOZYME WITH ADDED SIGNAL PEPTITUDE |

| | |
|---|---|
| | SIGNAL PEPTITUDE CHICKEN LYSOZYME |
| | ← → |
| | −Z-1 +1 +129 |
| Bacillas bravis CWP (B) | MKKRRVVNSYLLLLLASALALTVAPMAFA KVFGR...L |
| Chicken Lysozyme (C) | MRSLLILVLCFLPLAALG \vec{KV}FGR...L |
| Fruit fly lysozyme (F) | MKAFIVLVALACAAPAFG \vec{KV}FGR...L |
| Yeast Invertase (I) | MLLQAFLFLLAGFAAXISAWSNM \vec{KV}FGR...L |
| LSLP (L) | MRLLLLLLLLLPAALG \vec{KV}FGR...L |
| Honeybee selittis (M) | MKFLVNVALAFMVVYISYIYA \vec{KV}FGR...L |

As shown in FIG. 1, the pVL1393Lyz I is digested with the BamH I and Nhe I, and the signal sequence of yeast invertase and the upstream of the lysozyme gene were deleted. Meanwhile, the upstream sequences of the lysozyme having a signal sequence were synthesized with PCR, respectively. Each of the synthetic products was connected to the digested pVL1393Lyz I described above in order to construct a plasmid with a replaced signal sequence.

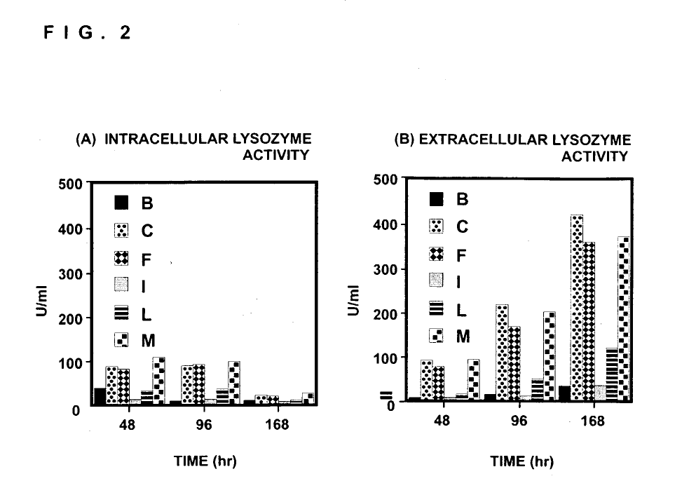

The obtained plasmid was introduced into an insect cell, and the intracellular representation and extracellular secretion were measured based on intracellular and extracellular lysozyme activities, respectively. The measurement results are shown in FIG. 2. Moreover, in FIG. 2, intracellular lysozyme activity (A) and extracellular lysozyme activity (B) are displayed as separate graphs, respectively.

As shown in FIG. 2(A), with respect to the intracellular lysozyme activity, hardly any activity could be discerned for those possessing a signal sequence deriving from yeast invertase (indicated by “I” in the diagram). Nevertheless, with the chicken lysozyme signal sequence (“C”) and fruit fly lysozyme signal sequence (“F”), activity approximately equivalent to the honeybee melittin signal sequence (“M”) used as the positive control was acknowledged, and the rise and fall of such activity also showed a similar pattern of decreasing with time. Further, although weak, intracellular activity could be acknowledged with the bacillus brevis CWP (“B”) and L8LP signal sequence (“L”).

Meanwhile, with respect to extracellular lysozyme activity, the chicken lysozyme signal sequence and fruit fly lysozyme signal sequence showed high activity similar to the honeybee melittin signal sequence (“M”), and the activity thereof elevated with time, and showed that this is symmetrical to intracellular activity. From this, it is clear that the chicken lysozyme signal sequence and fruit fly lysozyme signal sequence also elevate the representation of protein and enable the extracellular secretion of protein as with a honeybee signal sequence.

Example 2: Material for Examining Expression and Secretion of HIV glycoprotein (gp120)

In the following example, whether the expression/secretion can be elevated by replacing the signal sequence of the HIV-1Sf2rgp120, which is known to have considerably low expression and secretion, is examined. Thus, in the present Example, a material for performing such analysis was prepared.

(1) Preparation of Vector

First, the HIV-1SF2rgp120 is structured based on a baculovirus vector, and the SF2 signal sequence (SEQ ID NOS. 9 and 10) is supported at the gp120 of HIV-1 strain and at the upstream thereof. Moreover, the signal sequence to replace this SF2 signal sequence is shown in Table 2.

TABLE 2

| ORIGIN | SIGNAL PEPTITUDE |
|---|---|
| | ← → |
| | −29 −1 +1 |
| SF2-gp120 | MKVKGTRRRNYEHLWRWGTLLLGMLMICSA TEK... |
| (SF2) | |
| | −29 |
| SF162- | MRVKGIRKNYQHLWKRGGTLLLGMLMICSA VEK... |
| gp120 | |
| (SF162) | |
| −21 | |
| Honeybee | MKFLVNVALVFMVVYISYIYA APE... |
| melitin | |
| (HM) | |
| −18 | |
| Chicken | MRSLLILVLCFLPLAALG KVF... |
| lysozyme | |
| (cL) | |

That is, with respect to the signal sequence, in aforementioned Example 1, prepared among the signal sequences in which the promotion of expression and secretion was observed were the signal sequence deriving from chicken lysozyme (SEQ ID NO. 1) and newly the SF162 type HIV signal sequence (SEQ ID NO. 11). As the positive control, the aforementioned honeybee melittin signal sequence (SEQ ID NO. 7) was used.

Furthermore, the SF2 type HIV is directed toward the T cell, and the SF162 type HIV is directed toward the macrophage.

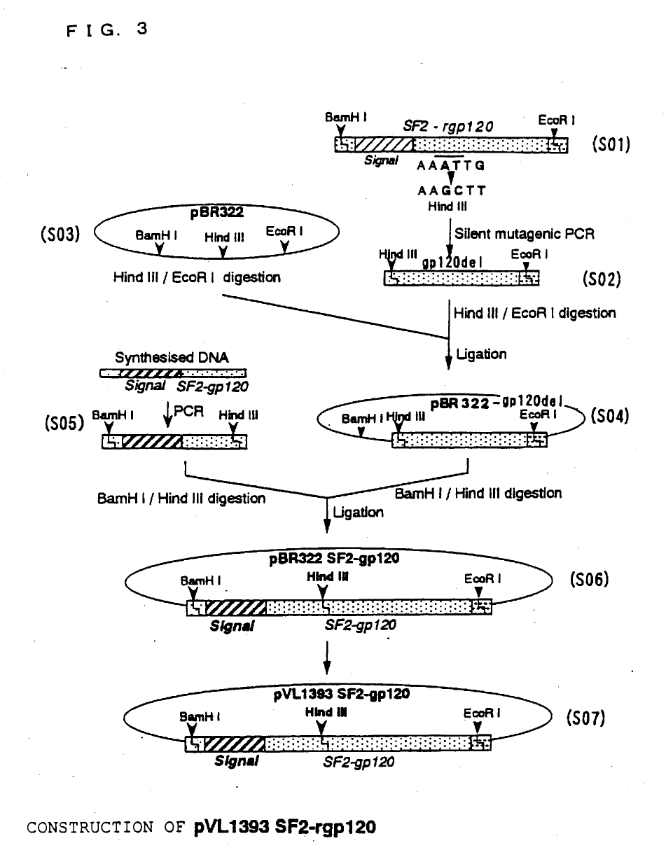

The structure of HIV-1SF2gp120 and the replacement procedures to the respective signal sequences described above are shown in FIG. 3.

As shown in FIG. 3, the recombinant gp120 (SF2-rgp120) having an SF2 signal sequence was used as the starting material. In this SF2-rgp120, a 2-base silent mutation is inserted on the gp120 sequence. Pursuant to a mutation method, this mutation restores the mutated base to obtain an SF2-gp120 (SEQ ID NO. 13, gp120 is shown in sequence 14), and forms the Hind III region (S01).

Thereafter, the SF2-gp120 was digested with Hind III and EcoR I, thereby abstracting the gp120 partially lacking an upstream sequence (gp120del), and eliminating the signal sequence (S02). Moreover, in order to recover this gp120del, a pBR322 digested with Hind III and EcoR I (S03) was prepared. The gp120 was connected to the prepared pBR322 in order to obtain a pBR322-gp120del.

Meanwhile, in order to insert the respective signal sequences into the upstream of the gp120, the respective signal sequences and DNA comprising an SF2 signal sequence in the upstream sequence of the gp120 were synthesized with a commercially available DNA synthesizer. PCR was further performed to this synthesized DNA in order to add a BamH I site to the upstream terminal and a Hind III site to the downstream terminal (S05).

This PCR fragment is thereafter inserted into the aforementioned pBR322-gp120del digested with the BamH I and Hind III in order to produce a pBR322SF2-gp120 (S06).

Finally, in order to the observe the expression inside the insect cell, the pBR322 is reconnected to with a baculovirus vector, thereby structuring a pVL1393SF2-gp120 (S07).

Pursuant to similar operations, the following were created: a pVL1393SF162-gp120 having an SF162 signal sequence, a pVL1393cL-gp120 having a chicken lysozyme signal sequence, and a pVL1393HMSF2-gp120 having a honeybee melittin signal sequence, which is the positive control.

Further, a baculovirus polyhedrin promoter (SEQ ID NO. 16) is supported by the aforementioned pVL1393, and this promoter enables the expression of the gp120 having the respective signal sequences.

(2) Preparation of Recombinant Baculovirus

Each of the aforementioned vectors is introduced, pursuant to the lipofectin method, into the AcPR23LacZ to which a LacZ gene of a cenocytic gene of a wild-type baculovirus has been introduced therein in order to create a recombinant baculovirus.

Two milliliters of an Sf21 cell of the insect cell (Invitrogen) (1.0 to 1.5×106 cells/ml) was wrapped around a 35 mm schale, left still at 27 degrees, and the cell was thereby affixed to the undersurface of the schale.

One hundred ng of AcRP23LacZ/Bsu36 I and 1 μl of each of the aforementioned vector DNA were injected into a microtube, and virus/DNA mixtures were prepared respectively by making the total volume 20 μl with distilled water added thereto. Next, 4 μl of lipofectin reagent and 4 μl of distilled water were mixed, left still for 15 minutes, and thereafter dropped into each of the foregoing virus/DNA mixtures.

The cells within the aforementioned schale were washed twice with a serum-free IPL-41 medium. After washing, each of the virus/DNA mixtures to which was added the foregoing lipofectin was dropped onto different schales, respectively, and incubated for 5 hours to overnight at 27° C. A 10% cow embryo serum-added IPL-41 medium was added to the respective schales, and incubated for an additional 3 to 4 days.

The incubated medium was recovered in a tube, and centrifuged at 2000 rpm for 5 minutes, and the rough virus solutions, which are supernatants, were respectively transferred to a new microtube, and preserved at 4° C.

The target virus (LacZ lacking virus) was refined with the plaque-assay method from these rough virus solutions.

A plurality of 60 mm schales having the SF21 cell affixed thereto were prepared, and the rough virus solutions gradually diluted from 10-3 to 10-9 were respectively added to different schales. After the addition, the schales were shaken every 10 minutes so as to make the infection uniform, and the infection was conducted for 1 hour.

After the infection, the supernatants were completely deleted, the IPL-41 agar medium (obtained by evenly mixing agros and IPL-41) was injected and solidified, and cultured for 3 to 5 days. After culturing, the X-gal added IPL-41 agar medium (30 μl of Xgal solution (20 mg/ml) dissolved in dimethyl sulfoxide per 2 ml of agar medium) was injected into the schale and solidified. This was cultured for 2 to 3 days thereafter, and the appearance of plaque was confirmed thereby.

Among the plaque that appeared, the plaque pursuant to the target virus takes on a white color from the LacZ deficiency, and the white plaque was recovered in a tube. A serum-free IPL-41 medium was injected into this tube, stirred intensely so as to elute the virus from the agar, and a refined virus solution was obtained thereby. Furthermore, it was confirmed that the target DNAs were respectively introduced into the refined recombinant virus pursuant to PCR.

Example 3: Expression and Secretion of gp120

Whether the expression/secretion of gp120 is affected by the difference in signal sequence by employing the recombinant virus prepared in aforementioned Example 2 was also examined.

Insect cell Sf21 was infected with the aforementioned recombinant virus under similar conditions, and each of the gp120s having the respective signal sequences was expressed. Four days after the virus infection, the cell-cultured solution was transferred to the likes of a test tube, centrifugal separation was performed thereto at 2000 rpm for 5 minutes, and the supernatants were recovered thereby. SDS polyacrylamide electrophoresis (SDS-PAGE) and the Western blotting method were conducted with the aforementioned supernatants, and the expression of each of the gp120 proteins was confirmed thereby.

In addition, the concentrating gel was set to 10% and the resolving gel was set to 7.5% with respect to the SDS polyacrylamide, and performed in accordance with common procedures. Moreover, an anti-HIVrgp120 sheep antibody (International Enzymes) was used as the primary antibody in the Western blotting method, and an HRP-labeled anti-sheep IgG rabbit antibody (ZYMED) was used as the secondary antibody, and performed in accordance with common procedures.

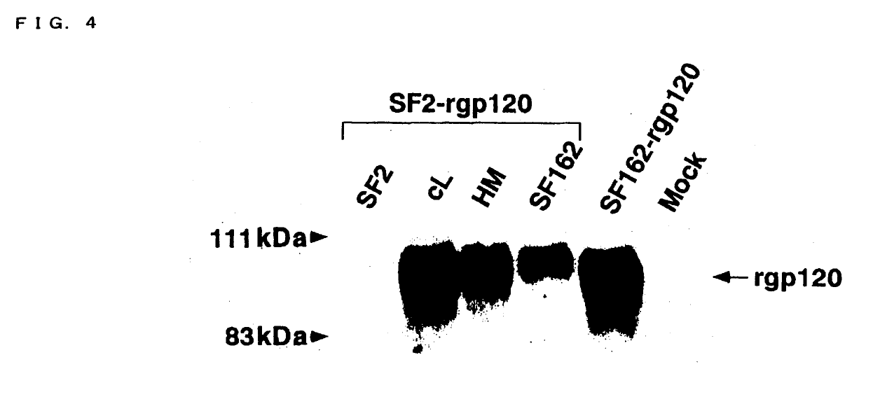

The results of Western blotting are shown in FIG. 4. As shown in FIG. 4, although expression cannot be observed with the gp120 having an SF2 signal sequence, it has been evidenced that the expression and secretion of gp120 becomes possible by replacing it with a signal sequence deriving from gp120 of chicken lysozyme (cL), honeybee melittin (HM) and SF162. Further, with respect to the expression and secretion promotion activity thereof, the signal sequence deriving from chicken lysozyme showed a higher activity than the signal sequence deriving from honeybee melittin, and the signal sequence deriving from the SF162gp120 showed approximately half the activity of the positive control.

Moreover, FIG. 4 shows the results upon the SF162-rgp120 using the vector to which is connected a recombinant gp120 deriving from SF162 at the downstream of the signal sequence of the SF162gp120. Further, the lane indicated as “mock” shows the negative control using a parent virus (AcPR23LacZ) to which a vector is not introduced.

Example 4: Confirmation of Glycosylation in GP120 Protein

Although a GP120 protein is originally a glycoprotein, whether the GP120 protein expressed and secreted according to Example 3 was sugar-modified was also examined.

In order to examine whether the gp120 protein was sugar-modified, deglycosylation processing was performed with N-glucosidase F for breaking the sugar chain at its root. This deglycosylation processing is described in detail below.

0.5% SDS-0.2 M2 mercaptoethanol (4 μl) was mixed with cultured supernatants containing the gp120 protein obtained pursuant to aforementioned Example 3, and heated at 100° C. for 3 minutes.

N glucosidase reaction solution (12 μl) was added to the heated mixture so as to make the total volume 20 μl. The composition of this N glucosidase reaction solution is 0.5 M phosphate buffer (pH8.5) (4 μl), 0.25 MEDTA (pH8.0) (0.8 μl), ¼ diluted TritonX-100 (0.8 μl), N-glucosidase F (20U/100 μl) (2 μl), and distilled water (4.4 μl).

After adding the aforementioned N glucosidase reaction solution, incubation was conducted at 37° C. for 24 hours and deglycosylation processing was performed thereafter. After deglycosylation, SDS-PAGE was conducted to the reaction solution with a 10% polyacrylamide gel. Further, as the positive control, similar processing was conducted by eliminating the N glucosidase from the N-glucosidase reaction solution.

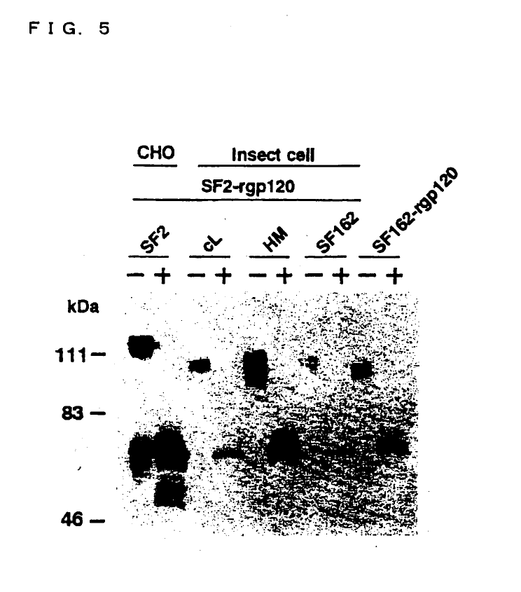

FIG. 5 shows the results upon dying the gel after having performed the SDS-PAGE. In FIG. 5, in the “-” lane, a non-deglycosylation processing sample processed by eliminating N-glycosidase was migrated, and in the “+” lane, a sample to which deglycosylation processing was performed was migrated. Further, in the lane indicated as “CHO”, a natural SF2gp120 glycoprotein obtained from the CHO cell infected with the SF2 type HIV was migrated so as to become the positive control of the gp120 deriving from an SF2 having a sugar chain.

As shown in FIG. 5, the gp120 protein (SF2-rgp120) deriving from SF2 shifts toward the side of the smaller molecular weight of the non-deglycosylation processing sample band, by performing the deglycosylation processing, whether having the chicken lysozyme signal sequence (cL) , honeybee melittin signal sequence (HM), or SF162 signal sequence. In other words, it has been evidenced that the molecular weight becomes smaller pursuant to the deglycosylation processing. The degree of band shift upon performing this deglycosylation processing showed a pattern approximately similar to a natural SF2-gp120 obtained from the infected CHO cell, which is the positive control.

Accordingly, by adding the signal sequences of chicken lysozyme, SF162 and honeybee melittin to the gp120 gene deriving from SF2, not only is the expression and secretion of the gp120 protein deriving from SF2 realized or promoted, it has been evidenced that it is possible to add a sugar chain similar to a natural glycoprotein.

From the above, it is clear that the expression systems having the signal sequence of the aforementioned chicken lysozyme, honeybee melittin or SF162 are capable of adding a sugar chain similar to a natural glycoprotein.

Example 5: Bioactivity of SF2 type gp120 Protein to which Each Signal Sequences is Added

The SF2 type HIV is directed toward and bonds with the CD4+T cell, and is a virus that attacks the CD4+T cell. Further, a natural SF2 type gp120 protein possesses a binding ability with the CD4.

As described above, whether the gp120 protein deriving from SF2 obtained by adding the respective signal peptides possesses a binding ability with the CD4 as with a natural SF2 type gp120 protein was examined through analysis employing a flow cytometry.

Foremost, the cultured supernatant (100 μl) containing the gp120 protein obtain in aforementioned Example 3 was added to the HeLa cell (1 to 5×105 cells) which expressed the recombinant CD4 (rCD4), and incubated at 37° C. for 30 minutes. After incubation, this was washed twice with PBS.

PE (phycoerythrin) conjugate OKT4 and anti-HIV human IgG were added thereto, and incubated at 4° C. for 1 hour. After incubation, this was washed twice with PBS.

Furthermore, FITC labeled anti-human IgG was added thereto, and incubated at 4° C. for 30 minutes. After incubation, this was washed twice with PBS.

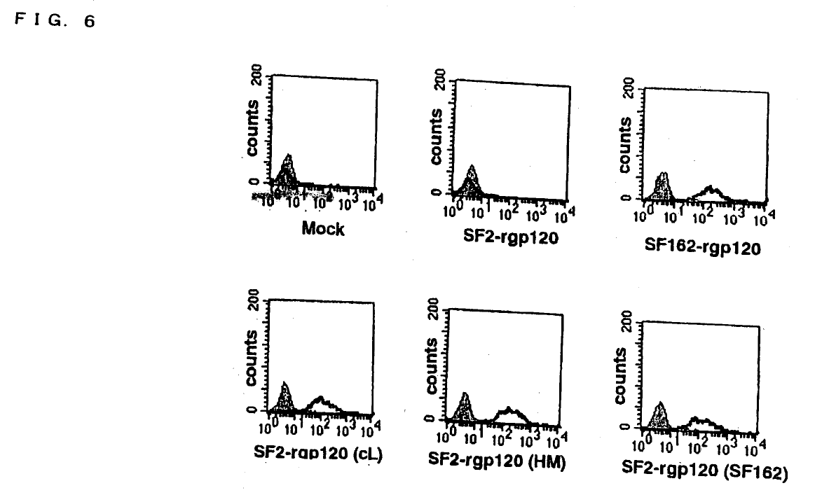

After washing, the cell distribution of cells that emit fluorescence and cells that do not emit fluorescence pursuant to FITC with flow cytometry was examined, and the binding ability of the SF2 type gp120 protein, in which the respective signal peptides were replaced, to the CD4 was analyzed. In other words, when the SF2-gp120 protein replaced with the respective signal peptides are bound with CD4 expression cells, the aforementioned HIV human antibody bonds with the cells via the gp120 protein. Then, the FITC labeled human IgG antibody bonds with the anti-HIV human antibody, and the cells emit fluorescence. Therefore, by examining the distribution of cells that emit fluorescence, it is possible to analyze the binding ability with the CD4. The results of flow cytometry are shown in FIG. 6.

Further, used as the positive control in this analysis were the cultured supernatant of cells (shown as SF2-rgp120 in FIG. 6) having introduced therein SF2-rgp120 (gp120 deriving from SF2 having an SF2 signal sequence) in which the expression of the gp120 could not be found in Example 3, and a cultured supernatant of insect cells to which a vector is not introduced (shown as “Mock” in FIG. 6). Moreover, a gp120 deriving from SF162 having an SF162 signal sequence was also used as the positive control.

As shown in FIG. 6, the fluorescent intensity of most cells with the negative control is low (100 to 101). Meanwhile, with the SF2-gp120 replaced with the chicken lysozyme signal sequence, honeybee melittin signal sequence and SF162 signal sequence, similar to the positive control, the fluorescent intensity of the cells increased (102 to 103). Accordingly, it has been evidenced that the gp120 protein expressed and secreted by the replacement of the aforementioned signal sequences possesses the bioactivity of a natural gp120 protein.

Thus, it has been evidenced that the expression system possessing the signal sequence of the aforementioned chicken lysozyme, honeybee melittin, or SF162 is capable of expressing and secreting a functional protein. This implies that the present expression system is effective in the production of protein capable of being used in protein preparation and vaccine manufacture. Particularly, the production of a gp120 protein deriving from SF2 having bioactivity will contribute significantly to the development and production of HIV vaccines.

Example 6: Influence on mRNA Expression

In the aforementioned Examples, the expression and secretion of the gp120 protein were promoted by adding the signal sequence deriving from chicken lysozyme, honeybee melittin or SF162 type HIV to the gp120 gene deriving from SF2. In this Example, however, examined was whether the promotion of this expression and secretion depends on the promotion of the translation process from mRNA to protein, or on the promotion of the transcriptional activity from DNA to mRNA.

The insect cell infected with the recombinant virus prepared according to Example 3 was used for this analysis. In addition, with respect to this recombinant virus, as described in Example 2 above, a gp120 gene deriving from SF2 with an added signal sequence deriving from chicken lysozyme (cL), honeybee melittin (HM) or SF162 type HIV (SF162) is inserted into the downstream of a baculovirus vector polyhedrin promoter.

The mRNA was recovered from each of these insect cells with the AGPA method.

Specifically, the cultured insect cells were recovered, frozen at −80° C., and, thereafter, transferred onto ice. Eight hundred μl of Solution D (prepared by adding 146.5 ml of distilled water to 125 ml of guanidine (iso) thiocyanate, 8.8 ml of 0.75 M sodium acid citrate (pH7), and 13.2 ml of 10% sarcosyl such that the total volume is 250 ml) was added to the cells, and the cells were immediately pulverized with a homogenizer.

After the pulverization of cells, 80 μl of 2M sodium acetate (pH4.0), and thereafter 800 μl of phenol and 160 μl of chloroform were added, and stirred for 10 seconds. After stirring, this was left still on ice for 15 minutes. After having left it still on ice, centrifugal separation was performed thereto at 4° C. (4000 rpm for 5 minutes) . Among the above, the aqueous phase was transferred to a new tube, and the nucleic acid was recovered thereby.

In order to refine the recovered nucleic acid, isopropanol was added to and mixed with the aqueous phase after recovery of the nucleic acid, left alone at −20° C. for 1 hour or more, and the sediments were recovered by centrifugal separation. The recovered sediments were completely dissolved by adding Solution D thereto. This isopropanol sedimentation was repeated thereafter.

After centrifugal separation, the recovered sediments were air-dried, and thereafter completely dissolved by adding 400 μl of purified water thereto. Refinement processing was performed to this solution with phenol/chloroform solution. Finally, ethanol sedimentation was performed, and the sediments were purely dissolved in order to obtain an RNA sample.

In order to confirm the RNA in the obtained RNA sample, electrophoresis and Northern hybridization were performed thereto.

Prior to conducting electrophoresis, the aforementioned sample was thermally denatured at 55° C. for 10 minutes, and thereafter left alone on ice for 3 minutes. Further, 5% agarose gel in which agarose is dissolved with a MOPS buffer solution was used as the electrophoretic gel.

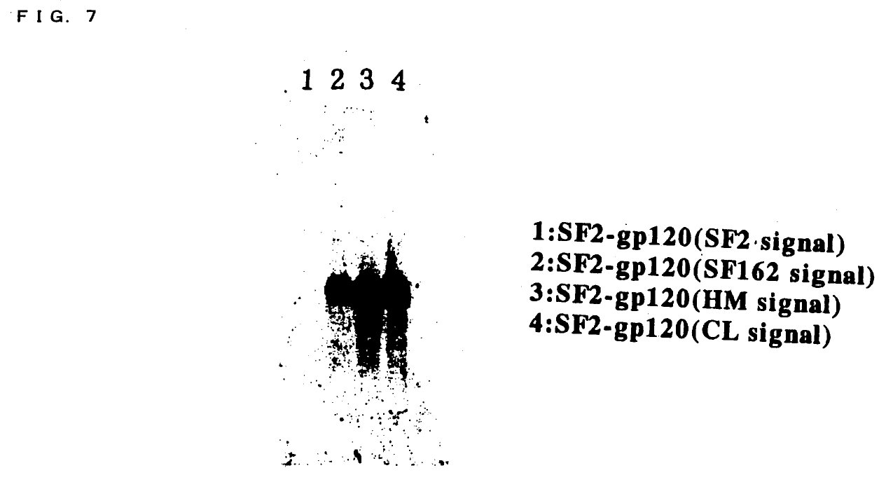

After electrophoresis, the migrated gel was affixed to the blotting film in order to transcribe the RNA in the gel onto the film. After transcription, a labeled probe produced with the gp120mRNA as the genetic template was used to perform Northern hybridization. The results of Northern blotting are shown in FIG. 7.

As shown in FIG. 7, with the SF2-gp120 (lane 1) having a signal sequence deriving from SF2, the expression of mRNA could not be observed at all. Meanwhile, with the SF2-gp120 having a signal sequence deriving from SF162 (lane 2), a signal sequence deriving from honeybee melittin (lane 3), and a signal sequence deriving from chicken lysozyme (CL), expression of mRNA was observed in each of the above signal sequences.

As described above, by adding a gp120 gene deriving from SF2 to a signal sequence deriving from SF162, a signal sequence deriving from honeybee melittin, or a signal sequence deriving from chicken lysozyme, it has been evidenced that the expression of mRNA in a gp120 gene deriving from SF2 comprising a signal sequence deriving from SF2 can be realized or promoted.

Thereby, from the foregoing result, it has been evidenced that one cause of the promotion of expression and secretion of a gp120 protein pursuant to these signal sequences lies in the promotion of the transcriptional level of mRNA.

Conventionally, a signal sequence was considered to be instrumental in modifications such as the post-translation transport of protein, addition of sugar chain, and so forth. Here, however, it has been shown that a signal sequence also has activity for promoting the transcription of mRNA. Not only does this imply that a signal sequence can be used for the purpose of promoting the expression and secretion of protein, this shows that it may also be used for promoting the transcription of mRNA. Particularly, in the present examples, the three types of signal sequences described above have been shown to have transcriptional promotion activity of gp120 deriving from SF2, and the promotion of lysozyme expression and secretion illustrated in Example 1 can also be considered as resulting from the promotion of the mRNA transcriptional activity. Thus, these signal sequences can be considered as being capable of promoting the mRNA expression of various genes including the SF2 type gp120 gene.

As described above, alternative embodiments of the signal sequence of the present invention are capable of implementing or promoting the expression of a gene including transcription, translation, post-translation protein secretion, modification such as glycosylation and so on by being added to a gene.

Further, by employing an expression vector possessing this signal sequence, the production of mRNA and expression/secretion of protein can be promoted, thereby facilitating the production of mRNA and the production of protein.

What is claimed is:

1. A nucleic acid inserted into the upstream of a gene linked expressibly to a promoter, wherein said nucleic acid is capable of implementing or promoting the expression of said gene from said promoter and wherein said nucleic acid is a secretory signal sequence of a chicken lysozyme or a secretory signal sequence of an SF162 type human immunodeficiency virus (HIV) gp120.

2. A nucleic acid according to claim 1, wherein the secretory signal sequence of said chicken lysozyme is the sequence shown in SEQ ID NO. 1.

3. A nucleic acid according to claim 1, wherein the secretory signal sequence of said SF162 type human immunodeficiency virus (HIV) gp120 is the sequence shown in SEQ ID NO. 11.

4. An expression cassette for implementing or promoting the expression of a gene, comprising: a promoter connected expressibly to said gene; a secretory signal sequence disposed upstream of said gene and expressed integrally with said gene from said promoter; and a 3′UTR disposed downstream of said gene, wherein said secretory signal sequence is a secretory signal sequence of a chicken lysozyme or a secretory signal sequence of an SF162 type human immunodeficiency virus (HIV) gp120.

5. An expression cassette according to claim 4, wherein the secretory signal sequence of said chicken lysozyme is the sequence shown in SEQ ID NO. 1.

6. An expression cassette according to claim 4, wherein the secretory signal sequence of said SF162 type human immunodeficiency virus (HIV) gp120 is the sequence shown in SEQ ID NO. 11.

7. An expression cassette according to claim 4, wherein said promoter and said 3′UTR derive from baculovirus polyhedrin.

8. A vector incorporating the expression cassette according to claim 4.

9. A vector according to claim 8, wherein said expression cassette is supported by a baculovirus vector.

10. An expression system for implementing the expression of a gene, wherein said expression system includes the vector according to claim 9, and an insect cell to which said vector is introduced.

11. An SF2 type HIVgp120 expression cassette, comprising: said SF2 type HIVgp120; a secretory signal sequence linked to the upstream of said SF2 type HIVgp120; a promoter connected to the upstream of said SF2 type HIVgp120 with said secretory signal sequence therebetween so as to express the SF2 type HIVgp120 to which said secretory signal sequence is linked; and a 3′UTR connected to the downstream of said SF2 type HIVgp120, wherein said secretory signal sequence is a secretory signal sequence of a chicken lysozyme or a secretory signal sequence of an SF162 type human immunodeficiency virus (HIV) gp120.

12. An SF2 type HIVgp120 expression cassette according to claim 11, wherein said promoter is a baculovirus polyhedrin promoter.

13. An SF2 type HIVgp120 expression cassette according to claim 11, wherein the secretory signal sequence of said chicken lysozyme is the sequence shown in SEQ ID NO. 1.

14. An SF2 type HIVgp120 expression cassette according to claim 11, wherein the secretory signal sequence of said SF162 type human immunodeficiency virus (HIV) gp120 is the sequence shown in SEQ ID NO. 11.

15. An SF2 type HIVgp120 expression vector incorporating the expression cassette according to claim 11.

16. An SF2 type HIVgp120 expression vector according to claim 15, wherein said expression cassette is supported by a baculovirus vector.

17. An SF2 type HIVgp120 expression cell retaining the SF2 type HIVgp120 expression vector according to claim 15.

18. An SF2 type HIVgp120 expression cell, wherein said SF2 type HIVgp120 expression cell is an insect cell retaining the SF2 type HIVgp120 expression vector according to claim 16.

19. An isolated SF2 type HIVgp120 protein produced from the cell according to claim 17.

20. A method for determining whether a secretory signal sequence, which is a test sample, is capable of implementing or elevating the expression of a gene, said method comprising: preparing an expression vector by connecting a monitor gene between a promoter and a 3′UTR on the vector, to which said secretory signal sequence is linked upstream so as to enable the expression from said promoter; introducing said expression vector into the cell; and measuring the transcriptional product of said monitor gene in said measurement step.

21. A determination method according to claim 20, wherein said promoter and said 3′UTR derive from baculovirus polyhedrin.

22. A system for determining whether a secretory signal sequence, which is a test sample, is capable of elevating the expression of a gene, said system comprising: (i) an expression vector which is connected to said gene and which has a promoter and a 3′UTR for connecting a monitor gene to which said secretory signal sequence is linked upstream so as to be expressible from said promoter and (ii) a cell for expressing said monitor gene by introducing said expression vector, wherein said system determines whether the nucleic acid, which is said test sample, is capable of elevating the expression of the monitor gene based on the representation of said monitor gene in said cell.

23. A system according to claim 22, wherein said promoter and said 3′UTR derive from baculovirus polyhedrin, and said cell is an insect cell.
